(19) United States
(12) Patent Application Publication
(10) Pub. No.: US 2008/0268047 A1
(43) Pub. Date: Oct. 30, 2008

(54) Controlled Release Multiple Layer Coatings

(75) Inventors: Tarun Kumar Mandal, Kenner, LA (US); Richard A. Graves, New Orleans, LA (US); Dakshinamurthy Devanga Chinta, Metairie, LA (US)

Correspondence Address: Adams and Reese LLP, 1221 McKinney Street, Suite 4400, Houston, TX, 77010, US

(73) Assignee: Xavier University of Louisiana, New Orleans, LA (US)

(21) Appl. No.: 11/739,219

(22) Filed: Apr. 24, 2007

(51) Int. Cl. A61K 9/24; A61K 33/12; A61K 38/12; B05C 11/00; A61K 9/16

(52) U.S. Cl. 424/472; 118/695

(57) ABSTRACT

A process for making controlled release pharmaceutical formulations is provided, which comprises supplying a plurality of solutions with syringe pumps for fluid bed coating, coating a substrate with a pH dependent soluble polymer solution, coating the polymer coated substrate with at least one layer of a solution of a therapeutically active substance and at least one layer of a second polymer solution, and alternating the layers so that the number, order, and volume of the layers controls the release of the therapeutically active substance. In alternate embodiments, the consecutively applied layers may contain coating materials, active ingredients or a mixture of coating materials and active ingredients; the layers can be applied in varying order. Also provided is a system for applying the coatings, wherein the syringe pumps are controlled by a computer in accordance with predetermined instructions.

### BACKGROUND OF THE INVENTION

I. Field of the Invention

The present invention relates generally to methods used to develop controlled release pharmaceutical formulations in which a plurality of coating solutions are applied consecutively to form a multiple layer coating.

II. Background and Prior Art

Solid pharmaceutical or other functionally active preparations which ensure a controlled release of active ingredients over a long period of time are well known in the art. These controlled release compositions are designed to contain higher concentrations of the active ingredient than conventional, immediate release dosage forms, and they are prepared in such a manner as to affect sustained or pulsed release of the active ingredient into the gastrointestinal digestive tract of humans or animals over an extended period of time. Well absorbed solid oral controlled release therapeutic drug dosage forms have inherent advantages over the conventional, immediate release drugs. These controlled release forms make it possible to reduce the number of doses of the drug to be administered daily, thereby facilitating patient compliance with the treatment plan prescribed by the physician. Moreover, they ensure a constant or elevated concentration of active ingredient in the body and a more sustained drug blood level response. By providing a controlled release of the drug over time, absorbed drug concentration spikes can be controlled or mitigated resulting in a smoother and more controlled blood level response. This also results in fewer side effects, reduced dangers of overdose, and economic advantages resulting from a more efficient dosage.

Many controlled release formulations, especially those in tablet and capsule form are provided with a coating that regulates the release of a therapeutically active substance. These formulations are characterized according to whether the active ingredient is released continuously with time, or is delivered as a series of pulses, separated by discrete periods of time during which the human or animal receives no active ingredient. The term controlled release as used herein, encompasses both continuous release and pulse release mechanisms. Various coating techniques have been utilized by the prior art to control the rate or the site of the release of the active ingredient in the pharmaceutical formulation.

Rate controlling layers can be made from polymers that prevent or slow the permeation of water into the substrate and various additives that alter permeability or that reduce drug diffusion through the coating. However, the prior art methods commonly used for coating substrates such as tablets, granules, pellets and other discrete particles do not allow easy manipulation of the layers as a means for controlling the delivery parameters of controlled release formulations. Hence, it is desirable to provide a process that can be used to easily and accurately adjust the delivery characteristics of controlled release pharmaceutical formulations.

Spray drying using fluid bed technology is well-known in the art and is commonly used for coating pharmaceutical formulations. Fluid bed coating machines may utilize a top spray system, a bottom spray system, also known as a Wurster Coater, or a combination of both. Spray drying generally involves supporting uncoated particles in a vertical column by injecting a continuous stream of air from the bottom of a column. A coating solution is atomized and sprayed onto the air-suspended particles, and the particles are then dried while supported by the air. The drying time of the applied coating can be regulated by controlling the atomization rate and/or the temperature of the supporting air stream. Also, the velocity of the air stream can be adjusted so that the air-suspended particles are maintained in a relatively confined region of the column. Moreover, the air stream is usually directed into one portion of the suspended bed of particles at a higher velocity than in the remaining portion of the fluidized bed. This causes the particles to flow upwardly in the portion of the bed subjected to the higher velocity air and induces a downward flow in the remaining portion of the bed to create a cyclical vertical movement of the particles within a generally central region of the column. The particles are repeatedly recirculated within the column, while successive layers of a coating solution are applied via aerosol spray until the desired thickness of that coating is formed. Each cycle of each coating consists of two phases: (1) spraying the substrate with polymer and (2) drying.

The prior art coating machines require an external device to supply the liquid coating solution to the fluidized bed machine. Typically, a peristaltic pump performs this function. U.S. Pat. No. 5,254,168, for example, discloses a fluid bed apparatus where a peristaltic pump supplies the liquid coating to the spray nozzles of the apparatus. However, the use of a peristaltic pump limits the application in a number of ways. First, a peristaltic pump only facilitates the application of a single liquid coating solution. In order to apply a plurality of coating solutions via the same spray nozzle, the parameters of the pump must be manually adjusted. Furthermore, the tubing that carries the coating solution must be physically replaced, especially if the various coating solutions cannot be intermixed due to their respective chemical properties. Having to manually adjust a peristaltic pump can be especially problematic during a product's research and development phase because it is necessary to repeatedly manipulate parameters such as the flow rate of the spraying solution, the polymer types, the volume and concentration of the polymer solutions, and the number and composition of the layers, in order to determine the ideal release properties for a given pharmaceutical formulation. Hence, it is desirable to provide a method that facilitates the manipulation of such parameters.

The present invention is directed to a new and novel coating method. The novel coating process of the present invention allows precise manipulation of the controlled release characteristics. As described hereinbelow, the coating process of the present invention has several advantages over coating processes described in the prior art or that are commercially used.

### SUMMARY OF THE INVENTION

Therefore, one object of the present invention is to provide a fluid bed coating process that facilitates the application of a plurality of coating layers.

It is also an object of the present invention to provide a coating process that allows effective manipulation of the release properties of a therapeutically active substance.

It is also an object of the present invention to provide an apparatus that facilitates fluid bed coating with a plurality of coating solutions wherein the parameters can be changed automatically.

A further object of the present invention is to streamline the manufacturing process of a time release pharmaceutical formulation by improving the accuracy of each coating.

Accordingly, the present invention provides a fluid bed drying process wherein a plurality of coating solutions are applied consecutively to the desired substrate using a plurality of syringe pumps to supply the plurality of coating solutions to the spray nozzle of the fluidized bed machine. The controlled release characteristics of the pharmaceutical formulation can be easily manipulated by changing the flow rate of the spraying solution, the polymer types, the volume, and concentration of the polymer solution(s), and the number and composition of the layers. The consecutively applied layers may contain coating materials, active ingredients or a mixture of coating materials and active ingredients.

### BRIEF DESCRIPTION OF THE DRAWINGS

For a further understanding of the nature and objects of the present invention, reference should be had to the following figures in which like parts are given like reference numerals and wherein:

FIG. 1 depicts a schematic diagram of the overall system employed to practice the present invention.

FIG. 2 depicts a dissolution profile resulting from the process of Example 1.

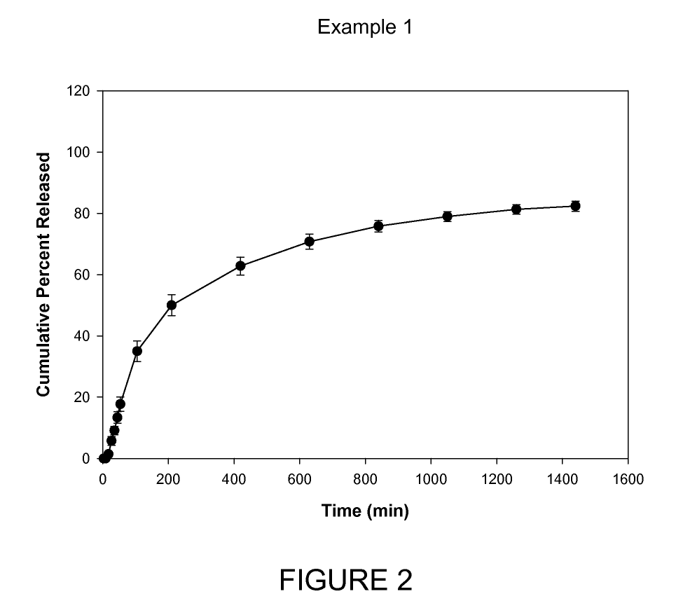

FIG. 3 depicts a dissolution profile resulting from the process of Example 2.

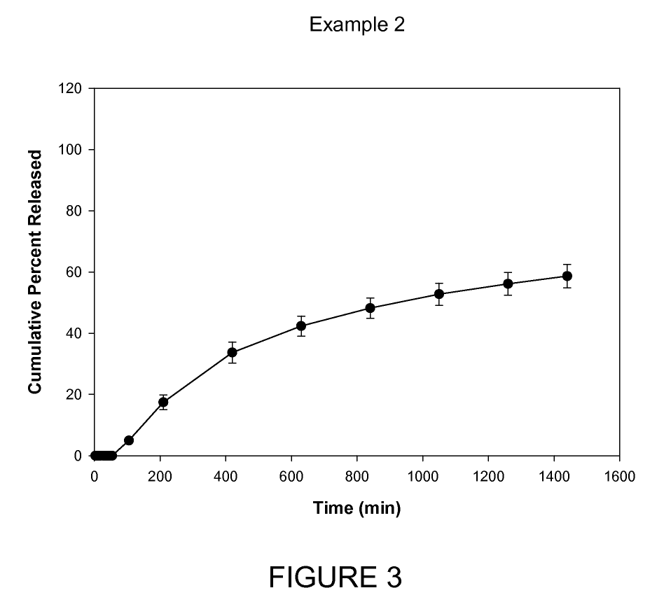

FIG. 4 depicts a dissolution profile resulting from the process of Example 3.

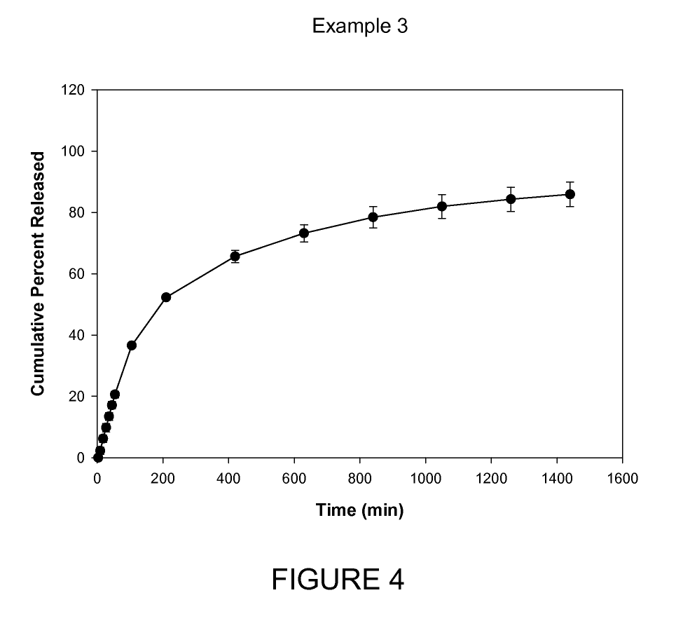

FIG. 5 depicts a dissolution profile resulting from the process of Example 4.

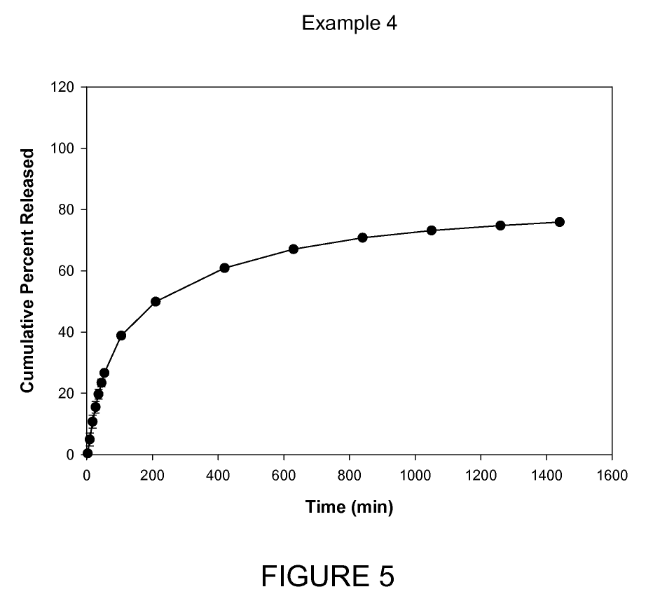

FIG. 6 depicts a dissolution profile resulting from the process of Example 5.

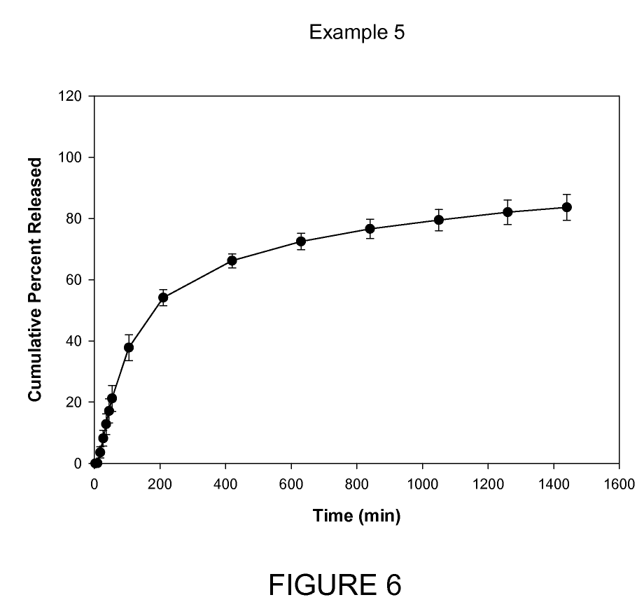

FIG. 7 depicts a dissolution profile resulting from the process of Example 6.

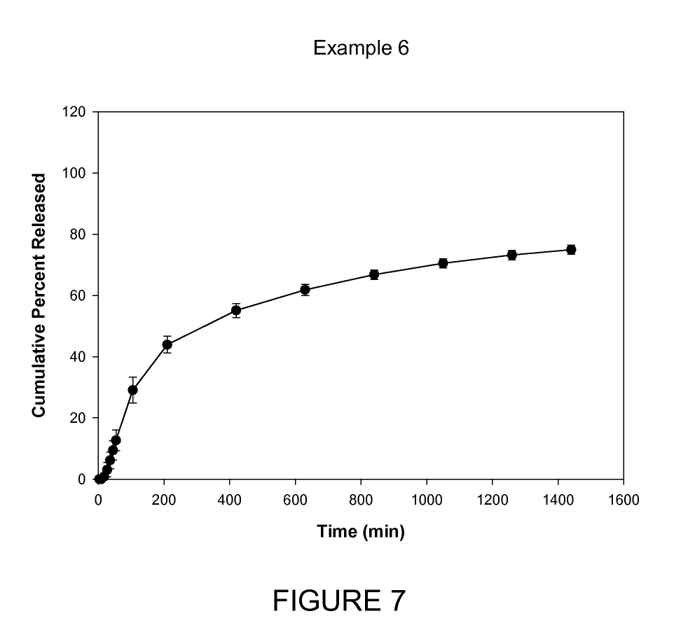

FIG. 8 depicts a dissolution profile resulting from the process of Example 7.

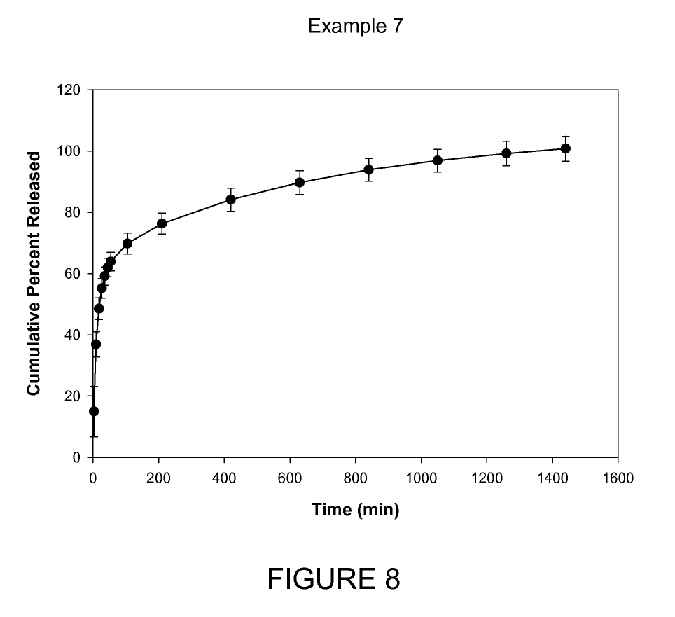

FIG. 9 depicts a dissolution profile resulting from the process of Example 8.

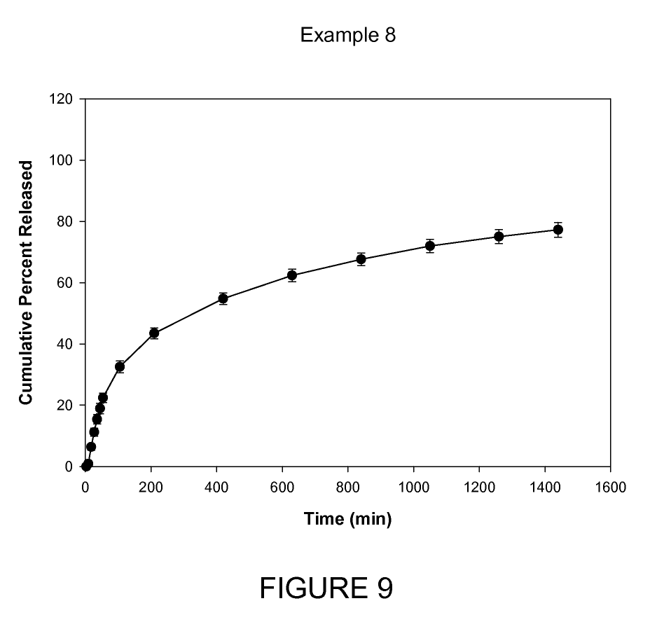

FIG. 10 depicts a dissolution profile resulting from the process of Example 9.

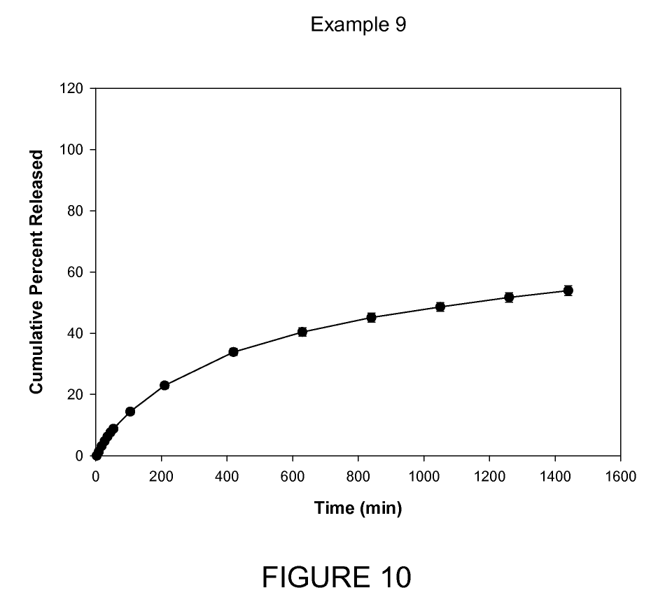

FIG. 11 depicts all of the dissolution profiles resulting from the processes of Examples 1-9 for comparison purposes.

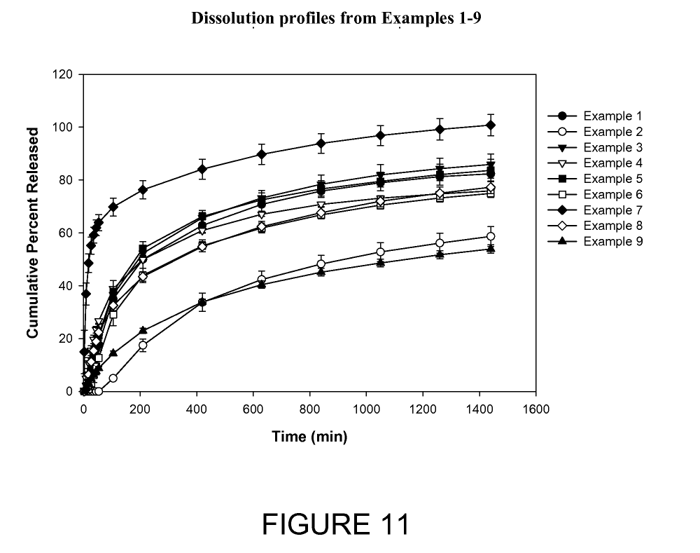

### DETAILED DESCRIPTION OF THE PREFERRED EMBODIMENTS

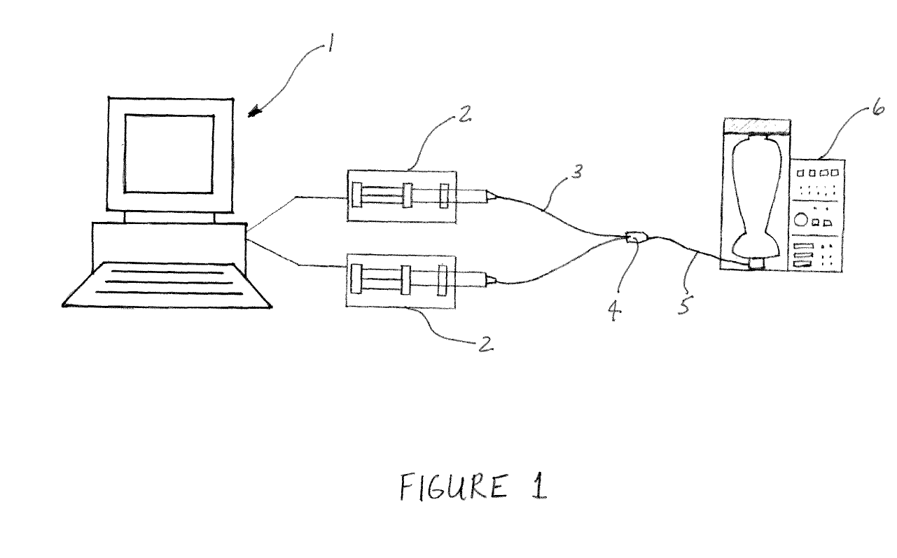

Turning now to FIG. 1, a schematic diagram of a preferred embodiment of the present invention is shown. A computer 1 controls a plurality of programmable syringe pumps 2. In the preferred embodiment, the tubing 3 is connected via a three-way connector 4 to another section of tubing 5 that is connected to the fluid bed dryer 6. As will be appreciated from the following description of the processes employed, any number of syringe pumps 2 may be used depending on the number of different coatings desired.

The coating process of the present invention may be used to coat various starting particles, or substrates such as tablets, seeds, pellets, beads, or other multi-particulate systems, in order to achieve the desired release properties for a given therapeutically active substance. In alternate embodiments, the therapeutically active substance may comprise a medicament or a macromolecule such as a peptide, protein, DNA or SiRNA. At least one layer of a pH dependent soluble polymer membrane is deposited on the surface of the starting particle. Additionally, at least one water insoluble controlled release layer, which reduces diffusion of the therapeutically active substance through the coating, is applied. There may also be a layer comprised of a hydrophilic, water swellable component. The water insoluble layer and/or the water swellable layers may contain the desired therapeutically active substance or alternatively, the active ingredient may comprise a further, separate layer. In the preferred embodiment the layers are applied consecutively. In alternate embodiments, the coating layers may be applied in any order.

In the preferred embodiment, the substrate is comprised of sugar spheres that are 60/80 mesh size. In alternate embodiments the particle mesh size can vary from about 12/14 to about 60/80 mesh. The pH dependent soluble polymer membrane is comprised of an acrylate solution and is applied to the substrate prior to the other layers. The water insoluble controlled release layer is comprised of an ethyl cellulose solution; in alternate embodiments, this layer may be comprised of other pharmaceutically acceptable water soluble, water swellable, and water insoluble polymers. In the preferred embodiment, the water swellable layer is comprised of a chitosan solution. In alternate embodiments low, medium, and high molecular weight chitosan may be used. In another embodiment, the water swellable layer may be absent.

### EXAMPLES

The following examples show how the release of a pharmaceutical formulation can be controlled by using a plurality of syringe pumps to manipulate the application of coating layers.

Prior to the coating of the sugar beads with drug and polymer, the beads were coated with an acrylate pre-coating. A MP Micro bench top fluid bed coater fitted with a bottom spray coater was charged with 50 grams sugar spheres NF (60/80 mesh size; Paulaur). The beads were coated with a Eudragit® L100 acrylate solution comprising 1.5 g acrylate, 0.5 g ethyl cellulose (dissolved in 30 ml ethanol 200 proof), 0.5 g talc, and 50 ml of a pH 7.4 50 mM potassium phosphate buffer solution using a peristaltic pump and fluid delivery line. After mixing, the pH of the solution is readjusted to 7.2 using sodium hydroxide. The coating conditions included inlet air temperature of 70° C., product temperature of 40° C., exhaust air temperature 38° C., atomizing air pressure 2 Bar, air velocity of 2.35-2.5 m/s, and a fluid delivery rate 2 ml/min. After coating, the beads were desiccated under vacuum for a minimum of 48 hours.

### Example 1

The bottom spray coater was charged with 20 grams of the acrylate coated beads. A propranolol drug solution was prepared by dissolving 2 grams of propranolol HCl (Sigma®) and 1 mg Cumarin dye (Sigma®) in 100 ml of 200 proof ethanol. The above mentioned drug solution was used to coat the specified amount of the sugar beads using the following conditions. The coating conditions included inlet air temperature of 60° C., product temperature of 30° C., exhaust air temperature 28±5° C., atomizing air pressure 2 Bar, air velocity of 2.35 m/s, and a fluid delivery rate 2 ml/min. After coating with the drug the beads were dried for 5 additional minutes in the coater at 60° C.

An ethyl cellulose overcoat solution was prepared by dissolving 4 g ethyl cellulose (DOW), 0.11 g dibutyl sebacate, and 1 mg Nile red dye in 160 ml 200 proof ethanol. The drug coated beads were then coated with the ethyl cellulose under the following conditions: inlet air temperature of 65° C., product temperature of 36° C., exhaust air temperature 34° C., atomizing air pressure 2 Bar, air velocity of 2.35 m/s, and a fluid delivery rate 1 ml/min. After coating, the beads were dried for an additional 10 minutes in the coater at 65° C.

### Example 2

Ethyl cellulose over coated propranolol sugar beads were produced in a similar manner as Example 1 with the following differences. The ethyl cellulose solution was prepared in 100 ml of ethanol instead of 160 ml, and the fluid delivery rate of the ethyl cellulose solution was 5 ml/min as opposed to 1 ml/min.

### Example 3

In this and all subsequent examples ethyl cellulose over coated propranolol sugar beads were formulated using a pulsed coating process to apply layers consecutively. To enable pulse coating a few modifications were made to the fluid delivery device of the fluid bed coating machine (MP Micro, Niro Inc., Columbia, Md.). First, the single peristaltic fluid delivery line was replaced by a single line branching into two lines, and the peristaltic pump was replaced by two controllable and programmable syringe pumps 2 as shown in FIG. 1 (NE1000, New Era Pump System, Farmingdale, N.Y.). The syringe pumps 2 were fitted with 60 ml syringes and the pumps 2 were connected to a computer 1 for automated control. Control of the pumps 2 was accomplished by sending a series of programming commands to the pumps 2. These commands controlled the flow rate of each pump 2, the total volume, and the total number of pump cycles. In addition, the computer 1 was used to control the withdrawing of fluid from the delivery tube 3 to eliminate mixing of the two components.

Drug and ethyl cellulose overcoat solutions were prepared as in Example 2. The syringes were filled with their respective drug and polymer solutions. The fluid bed coater was charged with 20 grams of acrylate coated sugar beads and set to the operating parameters as outlined in Example 1 with the exception that the fluid delivery of the propranolol and ethyl cellulose was delivered by the computer controlled syringe pump setup of FIG. 1 using the parameters outlined in Table 1.

TABLE 1

| Pump 1 (Drug Solution) | | | | Pump 2 (Ethyl cellulose Solution) | | | | |
|---|---|---|---|---|---|---|---|---|
| | | Total | | | | Total | | |
| | | Volume Per | | | | Volume | | |
| Flow Rate | | Cycle | Pause | Flow Rate | | Per Cycle | Pause | Total |
| ml/min | gm/min | ml | sec | ml/min | gm/min | ml | sec | Cycles |
| 2 | 0.04 | 20 | 120 | 5 | 0.2 | 20 | 120 | 5 |

### Example 4

Example 4 was produced in the same manner as Example 3 with the exception that the total volume of ethyl cellulose was 160 ml and the solution delivery program was changed to the values in Table 2.

TABLE 2

| Pump 1 (Drug Solution) | | | | Pump 2 (Ethyl cellulose Solution) | | | | |
|---|---|---|---|---|---|---|---|---|
| | | Total | | | | Total | | |
| | | Volume Per | | | | Volume | | Total |
| Flow Rate | | Cycle | Pause | Flow Rate | | Per Cycle | Pause | # of |
| ml/min | gm/min | ml | sec | ml/min | gm/min | ml | sec | Cycles |
| 2 | 0.04 | 20 | 120 | 1 | 0.025 | 32 | 120 | 5 |

### Example 5

Example 5 was produced in the same manner as Example 3 with the exception that the solution delivery program was changed to the values in Table 3.

TABLE 3

| Pump 1 (Drug Solution) | | | | Pump 2 (Ethyl cellulose Solution) | | | | |
|---|---|---|---|---|---|---|---|---|
| | | Total | | | | Total | | |
| | | Volume Per | | | | Volume | | |
| Flow Rate | | Cycle | Pause | Flow Rate | | Per Cycle | Pause | Total |
| ml/min | gm/min | ml | sec | ml/min | gm/min | ml | sec | Cycles |
| 2 | 0.04 | 20 | 120 | 1 | 0.04 | 20 | 120 | 5 |

### Example 6

Example 6 was produced in the same manner as Example 3. However, in place of having 5 uniform delivery cycles, the drug and coating was delivered in three different cycles as shown in Table 4.

TABLE 4

| Pump 1 (Drug Solution) | | | | Pump 2 (Ethyl cellulose Solution) | | | | |
|---|---|---|---|---|---|---|---|---|
| | | Total | | | | Total | | |
| | | Volume Per | | | | Volume | | |
| Flow Rate | | Cycle | Pause | Flow Rate | | Per Cycle | Pause | Total |
| ml/min | gm/min | ml | sec | ml/min | gm/min | ml | sec | Cycles |
| 2 | 0.04 | 50 | 120 | 5 | 0.2 | 25 | 120 | 1 |
| 2 | 0.04 | 25 | 120 | 5 | 0.2 | 25 | 120 | 1 |
| 2 | 0.04 | 25 | 120 | 5 | 0.2 | 50 | 120 | 1 |

### Example 7

In Example 7 the sugar beads were coated with successive layers of a solution of drug/ethyl cellulose in ethanol and a chitosan solution in 1% acetic acid.

The drug/ethyl cellulose solution was prepared by dissolving 2 g propranolol, 4 g ethyl cellulose, 0.11 g dibutyl sebacate, and 1 mg Nile red in 100 ml 200 proof ethanol. The chitosan solution was prepared by dissolving 0.5 g low molecular weight chitosan (Sigma®) in 100 ml of a 1% acetic acid solution. 0.5 g of talc was then dispersed in the solution.

The coating process involved charging the fluid bed coater with 20 g of acrylate coated sugar beads. Next the beads were coated with the drug/ethyl cellulose and chitosan solutions using the dual syringe pump setup of FIG. 1 and the following coating conditions. These included inlet air temperature of 70±5° C., product temperature of 36-44° C., exhaust air temperature 34-40° C., atomizing air pressure 3 Bar, and air velocity of 3.9 m/s. The fluid delivery parameters are listed in Table 5.

TABLE 5

| Pump 1 | | | | | | | | |
|---|---|---|---|---|---|---|---|---|
| (Drug/Ethyl cellulose Solution) | | | | Pump 2 (Chitosan Solution) | | | | |
| | | Total | | | | Total | | |
| | | Volume Per | | | | Volume | | |
| Flow Rate | | Cycle | Pause | Flow Rate | | Per Cycle | Pause | Total |
| ml/min | gm/min | ml | sec | ml/min | gm/min | ml | sec | Cycles |
| 2 | 0.12 | 20 | 120 | 2 | 0.01 | 20 | 120 | 5 |

### Example 8

In this example, the acrylate coated beads are first coated with the drug solution as in Example 1. Next the beads are coated with successive layers of ethyl cellulose solution (as in Example 3) and chitosan solution (as in Example 7). The process involved coating 20 g of drug-coated beads using the same operating parameters as those in Example 7 and using the fluid delivery program outlined in Table 6.

TABLE 6

| Pump 1 (Ethyl Cellulose Solution) | | | | Pump 2 (Chitosan Solution) | | | | |
|---|---|---|---|---|---|---|---|---|
| | | Total | | | | Total | | |
| | | Volume Per | | | | Volume | | |
| Flow Rate | | Cycle | Pause | Flow Rate | | Per Cycle | Pause | Total |
| ml/min | gm/min | ml | sec | ml/min | gm/min | ml | sec | Cycles |
| 2 | 0.08 | 20 | 120 | 2 | 0.01 | 20 | 120 | 5 |

### Example 9

Acrylate coated beads were prepared in a similar manner as Example 1 with the exception that the beads were dried in a desiccator for a minimum of 3 days. The beads were then treated with 40 ml of ethanol in fluid bed drier using the operating parameters as in Example 1. Again the beads were dried for 4 days. An ethyl cellulose solution was prepared by dissolving 4 grams of ethyl cellulose, 0.11 g dibutyl sebacate, and 1 mg Nile red in 100 ml ethanol. A drug/chitosan solution was prepared by dissolving 2 g propranolol and 0.5 g chitosan in 100 ml of a 1% acetic acid solution. 0.5 g talc was dispersed in the solution. The fluid bed coater was then charged with 20 g of the acrylate sugar beads and the coater was operated with the same parameters as in Example 7. The syringes were loaded with the ethyl cellulose and drug/chitosan solutions and the solutions were sprayed using the parameters outlined in Table 7.

TABLE 7

| Pump 1 (Drug/Chitosan) | | | | Pump 2 (Ethyl Cellulose) | | | | |
|---|---|---|---|---|---|---|---|---|
| | | Total | | | | Total | | |
| | | Volume Per | | | | Volume | | |
| Flow Rate | | Cycle | Pause | Flow Rate | | Per Cycle | Pause | Total |
| ml/min | gm/min | ml | sec | ml/min | gm/min | ml | sec | Cycles |
| 2 | 0.05 | 20 | 120 | 2 | 0.08 | 20 | 120 | 5 |

The dissolution rates of propranolol from various coated formulations of Examples 1-9 are shown in Table 8 below, and such results were obtained using the following procedures. The dissolution rates of propranolol from various coated formulations were studied using a VanKel automated dissolution apparatus (VK 7000, Varian Inc., Palo Alto, Calif.) connected to a UV-Viscible Spectrophometer (Carry 50 Tablet, Varian Inc., Palo Alto, Calif.). 900 ml pH 7.4 phosphate buffer was used as dissolution media and the temperature was maintained at 37±1° C. The USP II, rotating paddle method was used at a rotation speed of 50 rpm. A fixed volume of samples were automatically withdrawn through a filter at various pre set time intervals and analyzed for the concentration of propranolol using the automated spectrophotometer connected to the apparatus. The amount of propranolol dissolved at any time was measured at a wavelength of 296 nm.

TABLE 8
Dissolution time for various coated formulations.

| | | 50% Release | 60% Release | 80% Release |
|---|---|---|---|---|
| Example | Lag Time (min) | Time (min) | Time (min) | Time (min) |
| 1 | 18 | 240 | 360 | 1110 |
| 2 | 75 | 960 | 1440 | — |
| 3 | 6 | 195 | 330 | 840 |
| 4 | 6 | 210 | 390 | 2010 |
| 5 | 12 | 180 | 300 | 1260 |
| 6 | 21 | 300 | 570 | 2010 |
| 7 | 0 | 18 | 39 | 300 |
| 8 | 9 | 330 | 540 | 1680 |
| 9 | 6 | 1170 | 2010 | — |

As can be seen from the foregoing description of the preferred and alternate embodiments, the present invention is intended to provide a multiple layer coating for a controlled release formulation. Although the primary market for the product is for pharmaceutical applications, other applications may be developed for use in the field of probiotics and dietary supplements, as well as in the field of agriculture, e.g., fertilizers and insecticides. Although exemplary embodiments of the present invention have been shown and described, many changes, modifications, and substitutions may be made by one having ordinary skill in the art without necessarily departing from the spirit and scope of the invention.

What is claimed:

1. A process for making controlled release pharmaceutical formulations, comprising:
(a) providing one or more syringe pumps containing solutions for coating onto a substrate;
(b) coating the substrate with a layer of pH dependent soluble polymer solution;
(c) coating the polymer coated substrate with at least one layer of a solution of a therapeutically active substance and at least one layer of a second polymer solution; and
(d) alternating the layers in a manner that the number, order, and volume of the layers control the release of the therapeutically active substance during dissolution.

2. The process of claim 1, wherein the substrate is selected from the group consisting of tablets, beads and granules.

3. The process of claim 1, wherein the substrate comprises sugar spheres.

4. The process of claim 3, wherein the sugar spheres are between about 12/14 mesh size to about 60/80 mesh size.

5. The process of claim 1, wherein the pH dependent soluble polymer solution comprises an acrylate solution.

6. The process of claim 5, wherein the acrylate solution comprises Eudragit® L 100.

7. The process of claim 1, wherein the second polymer solution comprises a water insoluble polymer solution.

8. The process of claim 1, wherein the second polymer solution comprises a water soluble polymer solution.

9. The process of claim 8, wherein the water insoluble polymer solution comprises ethyl cellulose, dibutyl sebacate, red nile dye, and ethanol.

10. The process of claim 1, wherein the therapeutically active substance is selected from the group consisting of medicaments, peptides, proteins, DNA, SiRNA and mixtures thereof.

11. The process of claim 1, wherein the solution of therapeutically active substance comprises propranolol hydrochloride, acetic acid, and talc.

12. The process of claim 1, further comprising the step of coating the polymer coated substrate with a solution comprising a hydrophilic polysaccharide.

13. The process of claim 12, wherein the hydrophilic polysaccharide comprises chitosan.

14. The process of claim 13, wherein the chitosan is selected from the group consisting of low molecular weight chitosan, medium molecular weight chitosan, high molecular weight chitosan and mixtures thereof.

15. A process for making controlled release pharmaceutical formulations, comprising:
(a) providing one or more syringe pumps containing solutions for coating onto a substrate;
(b) coating the substrate with a layer of pH dependent soluble polymer solution;
(c) coating the polymer coated substrate with at least one layer of a second solution comprising a therapeutically active substance and a water insoluble polymer;
(d) coating the polymer coated substrate with another layer of a third solution comprising a hydrophilic polysaccharide; and
(e) alternating the layers in a manner that the number, order, and volume of the layers control the release of the therapeutically active substance during dissolution.

16. The process of claim 15, wherein the substrate is selected from the group consisting of tablets, beads and granules.

17. The process of claim 15, wherein the substrate comprises sugar spheres.

18. The process of claim 17, wherein the sugar spheres are between about 12/14 mesh size to about 60/80 mesh size.

19. The process of claim 15, wherein the pH dependent soluble polymer solution comprises an acrylate solution.

20. The process of claim 19, wherein the acrylate solution comprises Eudragit® L 100.

21. The process of claim 15, wherein the solution of therapeutically active substance comprises propranolol hydrochloride, ethyl cellulose, dibutyl sebacate, nile red dye, and ethanol.

22. The process of claim 15, wherein the hydrophilic polysaccharide comprises chitosan.

23. The process of claim 22, wherein the chitosan is selected from the group consisting of low molecular weight chitosan, medium molecular weight chitosan, high molecular weight chitosan and mixtures thereof.

24. A process for making controlled release pharmaceutical formulations, comprising:
(a) providing one or more syringe pumps containing solutions for coating onto a substrate;
(b) coating the substrate with a layer of pH dependent soluble polymer solution;
(c) coating the polymer coated substrate with a layer of ethanol;
(d) coating the ethanol coated substrate with at least one layer of a water insoluble polymer solution;
(e) coating the polymer coated substrate with another layer of solution comprising a therapeutically active substance and a hydrophilic polysaccharide; and
(f) alternating the layers in a manner that the number, order, and volume of the layers control the release of the therapeutically active substance during dissolution.

25. The process of claim 24, wherein the substrate comprises sugar spheres.

26. The process of claim 25, wherein the sugar spheres are between about 12/14 mesh size to about 60/80 mesh size.

27. The process of claim 24, wherein the pH dependent soluble polymer solution comprises an acrylate solution.

28. The process of claim 27, wherein the acrylate solution comprises Eudragit® L 100.

29. The process of claim 24, wherein the water insoluble polymer solution comprises ethyl cellulose, dibutyl sebacate, red nile dye and ethanol.

30. The process of claim 24, wherein the therapeutically active substance comprises propranolol.

31. The process of claim 24, wherein the therapeutically active substance comprises a solution of propranolol hydrochloride, chitosan, acetic acid, and talc.

32. The process of claim 24 wherein the therapeutically active substance is selected from the group consisting of medicaments, peptides, proteins, DNA, SiRNA and mixtures thereof

33. The process of claim 24, wherein the hydrophilic polysaccharide comprises chitosan

35. A process as in claims 31 or 33, wherein the chitosan is selected from the group consisting of low molecular weight chitosan, medium molecular weight chitosan, high molecular weight chitosan and mixtures thereof.

36. The process of claim 24 further comprising the step of desiccating the substrate after coating with ethanol and before coating with consecutive layers.

37. A process as in claims 1, 15, or 24, further comprising the step of desiccating after coating with a pH dependent soluble polymer solution and before coating with consecutive layers.

38. A process as in any one of claims 1, 15, and 24, in which the coating of at least one layer is achieved with pulsed release.

39. A system for manufacturing controlled release pharmaceutical formulations, comprising:
(a) a fluid bed coating machine for delivering and applying one or more coatings to a substrate;
(b) a plurality of controllable syringe pumps containing solutions to be applied to the substrate as the coatings;
(c) a convergent conduit fluidically connected between the syringe pumps and the fluid bed coating machine, wherein the solutions from the syringe pumps are delivered to the fluid bed coating machine; and
(d) a computer operatively connected to the syringe pumps, wherein the computer provides control signals to the syringe pumps in accordance with predetermined instructions.
